(19) United States
(12) Patent Application Publication
(10) Pub. No.: US 2023/0100707 A1
(43) Pub. Date: Mar. 30, 2023

(54) MELANOMA THERAPEUTICS

(71) Applicant: ESCAPE THERAPEUTICS, INC., Turlock, CA (US)

(72) Inventor: Basil M. Hantash, Turlock, CA (US)

(21) Appl. No.: 17/799,201

(22) PCT Filed: Feb. 16, 2021
(86) PCT No.: PCT/US2021/018247

§ 371 Date: Aug. 11, 2022

Related U.S. Application Data
(60) Provisional application No. 62/976,255, filed on Feb. 13, 2020.

(51) Int. Cl. C07K 7/06; C07K 5/097; A61P 35/00

(52) U.S. Cl. CPC C07K 7/06; C07K 5/0821; A61P 35/00; A61K 38/00

(57) ABSTRACT

Two therapeutics, oligopeptides P5 and P14, which have proven to be selectively cytotoxic towards multiple melanoma cell lines in vitro. These oligopeptides offer an efficacious treatment for all types of pathogenic melanocytes and stages of melanoma, and a cost effective prophylactic to all individuals who are at high risk of developing skin cancer. In addition, these oligopeptides can be formulated into a sunscreen to serve as a prophylactic against any precancerous lesion or dysplastic nevi that might develop in high risk groups.

### Cross-Reference to Related Application

This application claims priority to U.S. patent application 62/976,255, filed Feb. 13, 2020, which is incorporated by reference along with all other references cited in this application.

### BACKGROUND OF THE INVENTION

This invention relates to therapeutics for melanoma.

The incidence of malignant melanoma (MM) has been increasing at an alarming rate worldwide, resulting in a major public health problem in the United States. In 2019, 96,480 new cases of melanoma are expected to be diagnosed. The American Cancer Society estimates melanoma will be the cause of 7230 deaths in 2019.

In the United States alone, direct costs of melanoma morbidity and mortality are estimated to reach over $8.1 billion in 2019. This does not include the incalculable psychosocial impact on family members as well as the overall loss of productivity given melanoma's ability to afflict younger individuals. With surgery being the primary treatment option for melanoma, the psychosocial impact on the patient is unfathomable. Exposure to solar UV radiation, fair skin, dysplastic nevi syndrome, and a personal or family history of melanoma are major risk factors for melanoma development. To date, there are no treatments offered for high risk groups other than recommendations of avoiding sun exposure, the use of sunscreen with an SPF of 15 or higher and regular examination for any skin abnormalities.

In contrast to the highly effective treatment options for nonmelanoma skin cancer (NMSC) with a curative approach by surgery in the majority of cases, the therapeutic efficacy in melanoma utterly depends on early recognition of superficial tumors, which highlights the basic importance to advance with better strategies and more efficacious therapeutics that are inspired by our growing knowledge and understanding of the molecular behavior of malignant tumors and tumorigenesis.

Therefore, there is a need for improved therapeutics for melanoma.

### BRIEF SUMMARY OF THE INVENTION

Two therapeutics, oligopeptides P5 and P14, which have proven to be selectively cytotoxic towards multiple melanoma cell lines in vitro. These oligopeptides offer an efficacious treatment for all types of pathogenic melanocytes and stages of melanoma, and a cost effective prophylactic to all individuals who are at high risk of developing skin cancer. In addition, these oligopeptides can be formulated into a sunscreen to serve as a prophylactic against any precancerous lesion or dysplastic nevi that might develop in high risk groups.

Other objects, features, and advantages of the present invention will become apparent upon consideration of the following detailed description and the accompanying drawings, in which like reference designations represent like features throughout the figures.

### BRIEF DESCRIPTION OF THE DRAWINGS

FIG. 1 shows oligopeptides P5, P14, and P15.

FIG. 2 shows the cytotoxic effects of oligopeptides relative to the positive control HQ after a 72-hour incubation using the MTT cell proliferation and viability assay.

FIG. 3 shows the cytotoxic effects on human melanoma cells.

FIGS. 4-9 show the cytotoxic effects that P5, P14, and HQ exert on mouse and human melanoma cells.

FIGS. 10-12 show the selective cytotoxicity of P5 and P14 compared to the indiscriminate toxicity of HQ towards healthy normal skin cells like epidermal keratinocytes in FIG. 10, melanocytes in FIG. 11, and fibroblasts in FIG. 12.

### DETAILED DESCRIPTION OF THE INVENTION

### Introduction

The incidence of malignant melanoma (MM) has been increasing at an alarming rate worldwide, resulting in a major public health problem in the United States. In 2019, 96,480 new cases of melanoma are expected to be diagnosed. The American Cancer Society estimates melanoma will be the cause of 7230 deaths in 2019. In the United States alone, direct costs of melanoma morbidity and mortality are estimated to reach over $8 billion in 2019. This does not include the incalculable psychosocial impact on family members as well as patients managing the uncertainty and anxiety of surgery, the primary treatment option in most cases. Furthermore, there is a substantial loss of productivity given melanoma's ability to afflict younger individuals. Melanoma originates in melanocytes, most commonly in the skin. Exposure to ultraviolet radiation (UVR) appears to be the greatest inducer of melanoma through many mechanisms, including suppression of the skin immune system, induction of melanocyte cell division, free radical production, and damage of melanocyte DNA.

The risk of melanoma developing in people with a history of sunburns is double that in those without such history. Moreover, the age at which sunburns occur appears to be important with those occurring in childhood conveying the highest risk. Patients with xeroderma pigmentosum (a genetic disorder characterized by deficient DNA repair mechanisms and a corresponding hypersensitivity to UVR damage) have a substantially higher risk of developing melanoma, particularly in sun-exposed skin. Other sources of non-solar UVR, such as tanning beds and sun lamps, have been linked to increased risk of developing cutaneous melanoma.

In approximately 25 percent of cases, melanoma occurs in conjunction with a preexisting nevus. The risk is approximately 1.5-fold higher in people with 11 to 25 nevi (compared with less than or equal to 10 nevi) and doubled with every increase of 25 nevi. In contrast to the highly effective treatment options for non-melanoma skin cancer (NMSC) where surgical cure is achieved in the majority of cases, the therapeutic efficacy in melanoma depends on early recognition of superficial tumors, highlighting the importance of early detection and the need more efficacious treatment options.

Over the past decade, improved knowledge of the mitogen-activated protein kinase (MAPK) pathway and its implication in cancer development has led to attempts to target mutated forms of oncogenic kinases such as B-RAF, extracellular-signal-regulated kinases (ERK), and MAPK/ERK kinase (MEK). B-RAF is a member of the RAF kinase family, which acts in the MAPK/ERK signaling pathway responsible for regulating cellular proliferation, differentiation, and survival. Of the three RAF kinases (A-RAF, B-RAF and C-RAF), only B-RAF is frequently mutated in various cancers. A single base missense substitution (T to A at nucleotide 1,799), that changes valine to glutamic acid at codon 600 (V600E) in exon 15, accounts for 90 percent of all BRAF mutations within melanoma tumors. The consequence of this mutation is a 500-fold increase in kinase activity relative to the wildtype form, leading to constitutive upregulation of ERK signaling and subsequent unregulated cell growth, cell transformation, and eventual tumor initiation.

To date, there are no preventative treatments offered for high risk groups other than recommendations of avoiding sun exposure, the use of sunscreen with an SPF of 15 or higher and regular examination. To meet this urgent unmet medical need, we have screened a series of novel oligopeptides with high affinity for both kinases B-RAFV600E and C-RAF using molecular modeling in silico and compared them to the known kinase inhibitors sorafenib and PLX4032.

Herein we describe the identification of two oligopeptides, P5 and P14, which have proven to be selectively cytotoxic towards multiple melanoma cell lines in vitro. Our ultimate goal will be to offer an efficacious treatment for all stages of melanoma, and a cost effective prophylactic to all individuals who are at high risk of developing skin cancer. In addition, these oligopeptides can be formulated into a sunscreen to serve as a prophylactic against any precancerous lesion or dysplastic nevi that might develop in high risk groups.

### Materials and Methods

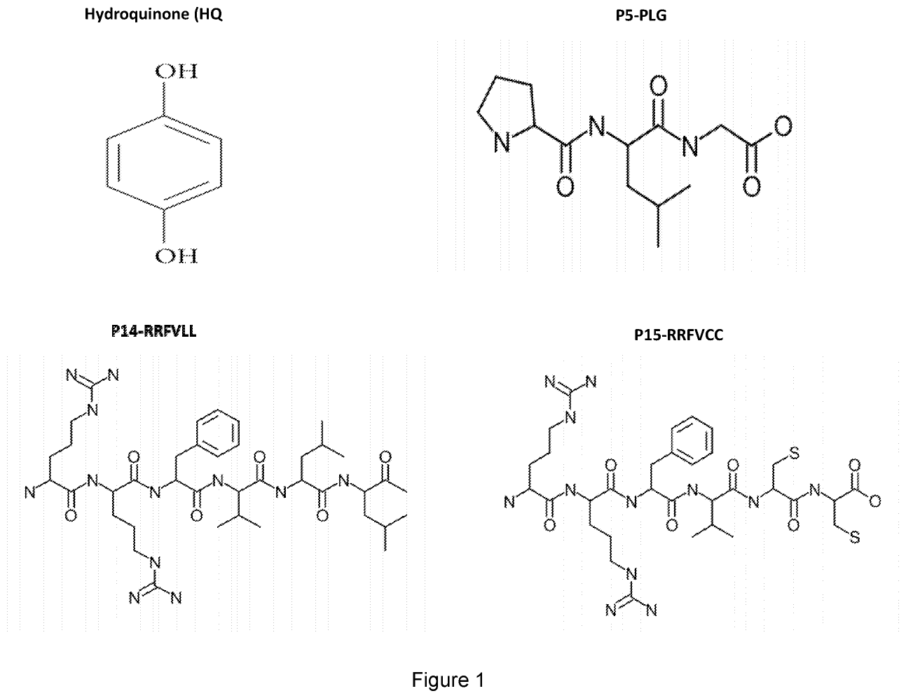

Oligopeptides P5, P14, and P15 (FIG. 1) were synthesized by Bio Basic, Inc. (Ontario, Canada) using solid-phase FMOC chemistry.

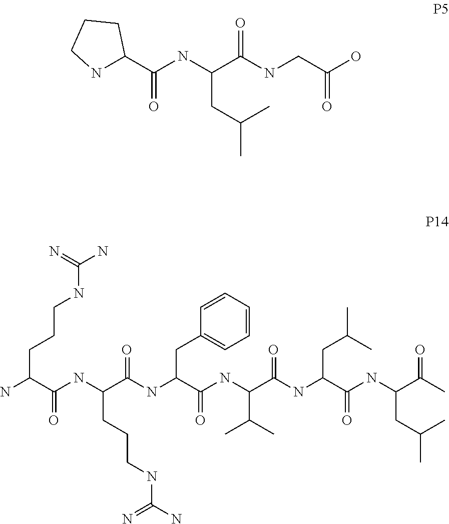 [SMILES: CC(C)CC(NC(=O)C1CCCN1)C(=O)NCC(=O)O ; CC(=O)C(CC(C)C)NC(=O)C(CC(C)C)NC(=O)C(NC(=O)C(Cc1ccccc1)NC(=O)C(CCCNC(=N)N)NC(=O)C(N)CCCNC(=N)N)C(C)C]

Hydroquinone was purchased from Sigma-Aldrich (St. Louis, Mo.).

Molecular modeling of BRAFV600E and CRAF inhibitors

Enzyme 3-D X-ray crystallography structures of wild type B-RAF (PDB ID: 1UWH, chain A), mutated B-RAFV600E (PDB ID: 3C4D, chain A) and C-RAF (PDB ID: 3LB7, chain A) were obtained from PDB and the oligopeptides were docked into the ATP binding pocket of the enzyme using AutoDock version 4.2.2.1 and AutoDock Vina version 1.0.3. The enzymes were prepared for docking by replacing missing hydrogens, removing water, NO3+, and Ca2+ions, adding Kollman charges and computing Gasteiger charges using Swiss-Pdb Viewer version 4.0.1 and AutoDockTools version 1.5.4. Sorafenib and PLX4032 structures were obtained from the PubChem Compound Database. Peptide ligands were created using PyMOL version 1.1 beta3 or Avogadro version 1.0.0 and saved as “.pdb” files. The ligands were then processed using the Dundee PRODRG2 server and AutoDockTools to determine charges and torsions. Each peptide was allowed all possible torsions. The binding modes of these inhibitors to B-RAFV600E and C-RAF were analyzed through molecular docking to derive structure-activity relationships.

### Cell Culture

Human and mouse melanoma cell lines as wells as Fibroblasts were cultured using DMEM with L-glutamine, 10 percent FBS and 1 percent P/S (Thermo Fisher Scientific, NY). Human melanocytes were cultured using medium 254 (Thermo Fisher Scientific, NY) and incubated at 37 degrees Celsius in a humidified 5 percent CO₂ chamber.

Human neonatal epidermal keratinocytes were cultured in Epilife media containing 60 μM of calcium chloride (Thermo Fisher Scientific, NY). All cell cultures were incubated in a humidified chamber at 37 degrees Celsius and 5 percent CO₂.

### Viability or Proliferation and Cytotoxicity Assays

Proliferation rates were determined using a TACS® MTT Cell Proliferation Kit (R&D Systems, Minneapolis, Minn.). Cells were seeded at 2.5×10 ⁴ per well in 96-well plates in a humidified atmosphere with 5 percent CO₂ at 37 degrees Celsius. Twenty-four hours later, oligopeptides or hydroquinone were added to the corresponding wells at varying concentrations (0.03, 0.1, 0.3, 1, and 3 millimolar (mM)), and cultures were incubated for 72 hours. The remainder of the procedure was performed following the manufacturer's protocol.

### Cellular toxicity

Cellular toxicity was measured using a trypan blue dye exclusion assay. Cells were cultured in 6-well plates at a density of 4×10⁵ cells per well. Each well received a different concentration of oligopeptide or HQ (0.03, 0.1, 0.3, 1, and 3 millimolar). Plates were incubated at 37 degrees Celsius in a humidified 5 percent CO₂ chamber. After 24 hours, an aliquot was taken and cells counted using a hemacytometer. Cytotoxicity was measured according to the following formula:

$$[ \underline{1 - ( ( number of cells in control - number of live cells in test sample} ) ] \times 100 \% ( number of cells in control )$$

The same procedure was repeated after 3 and 6 days of incubation.

### Results:

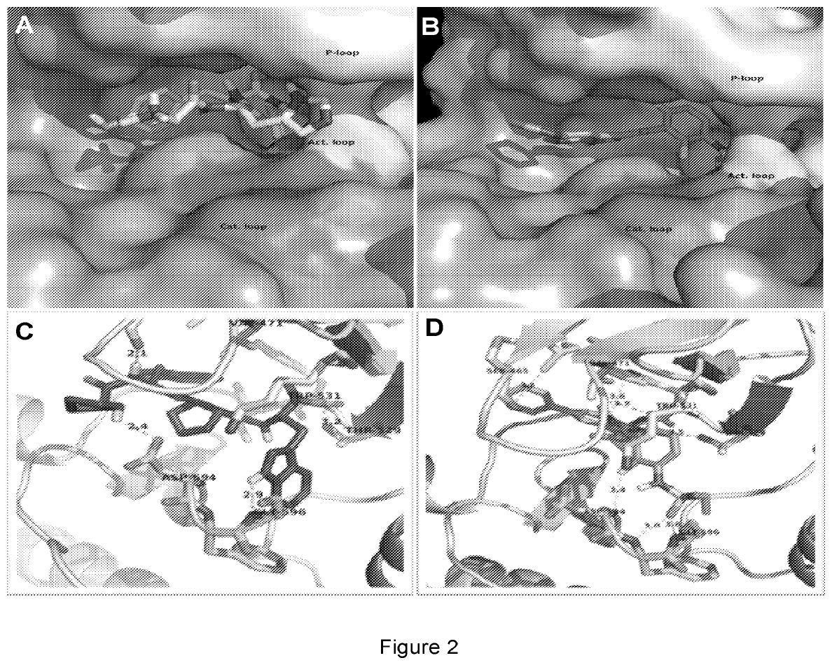

PLX4032 and sorafenib served as benchmarks for in silico screening of our internally generated oligopeptide library. The region (activation loop) encompassing the V600E mutation in B-RAF, which consequently induces the conformational change and hyperactivation of the kinase, is highlighted in green (FIG. 2A and 2B). FIG. 2 also shows that despite the significant interaction similarities between oligopeptide P14 (FIG. 2A) and PLX4032 (FIG. 2B) with the ATP binding pocket, P14 demonstrated a closer interaction with the activation loop. P14 formed several hydrogen bonds, which in combination with hydrophobic interactions pulled it to within 2-3 Angstroms from important residues in the ATP binding pocket (FIG. 2C) versus 3-4 Angstroms for PLX4032 (FIG. 2D).

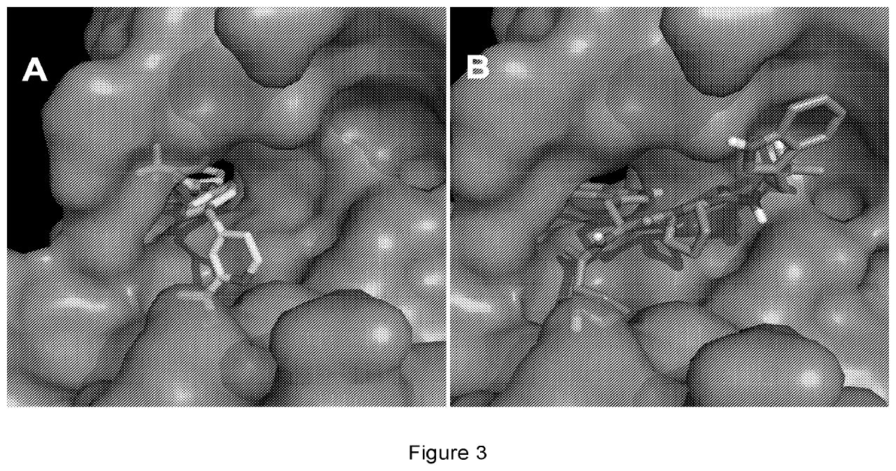

FIG. 3 illustrates the molecular docking interaction of P14 and sorafenib with the ATP binding pocket of C-RAF. P14 exhibited favorable conformations and enhanced binding to the catalytic pocket (FIG. 3B) relative to that demonstrated by sorafenib (FIG. 3A). This advantage is facilitated by favorable hydrogen bonding and hydrophobic interactions. Our preliminary molecular modeling data suggest that, relative to the 2 benchmarks sorafenib and PLX4032, the proposed oligopeptides possess comparable or improved affinity to both mutated B-RAF and wildtype C-RAF, justifying their further study as potential development candidates.

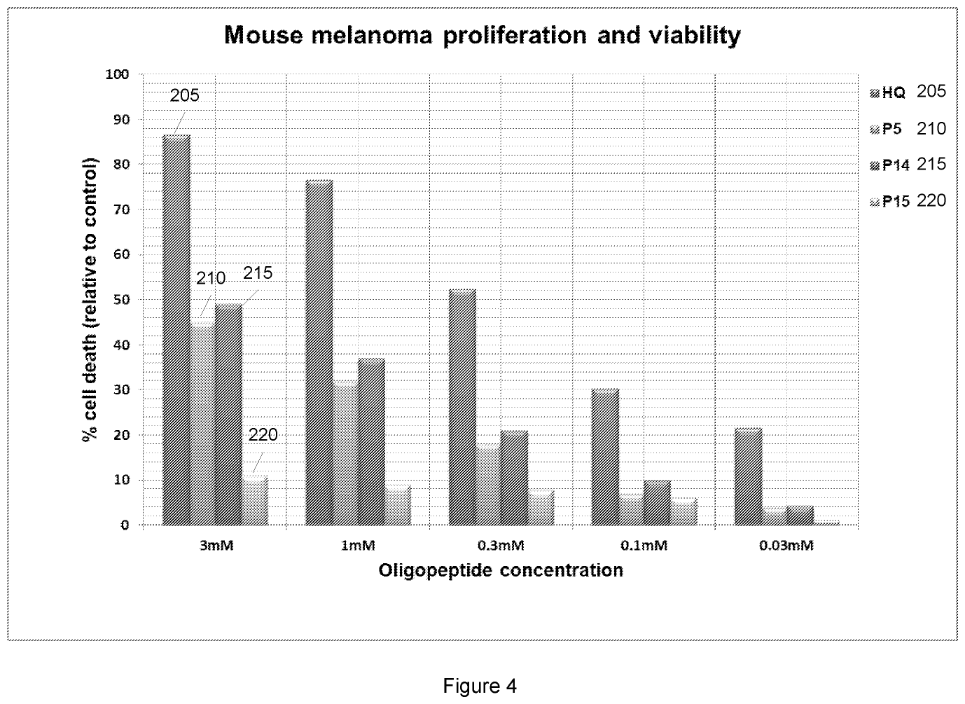

We next performed MTT assays to test for oligopeptide effects on the viability and proliferation of mouse melanoma cells and human metastatic melanoma line previously shown to harbor the V600E BRAF mutation. FIG. 4 shows that incubation for 3 days with 3 millimolar P5 or P14 resulted in 44 percent or 49 percent cell death, respectively, compared to 90 percent cytotoxicity for HQ after same period of incubation. Oligopeptide P15 showed only minor cytotoxic effects after 3-d incubation.

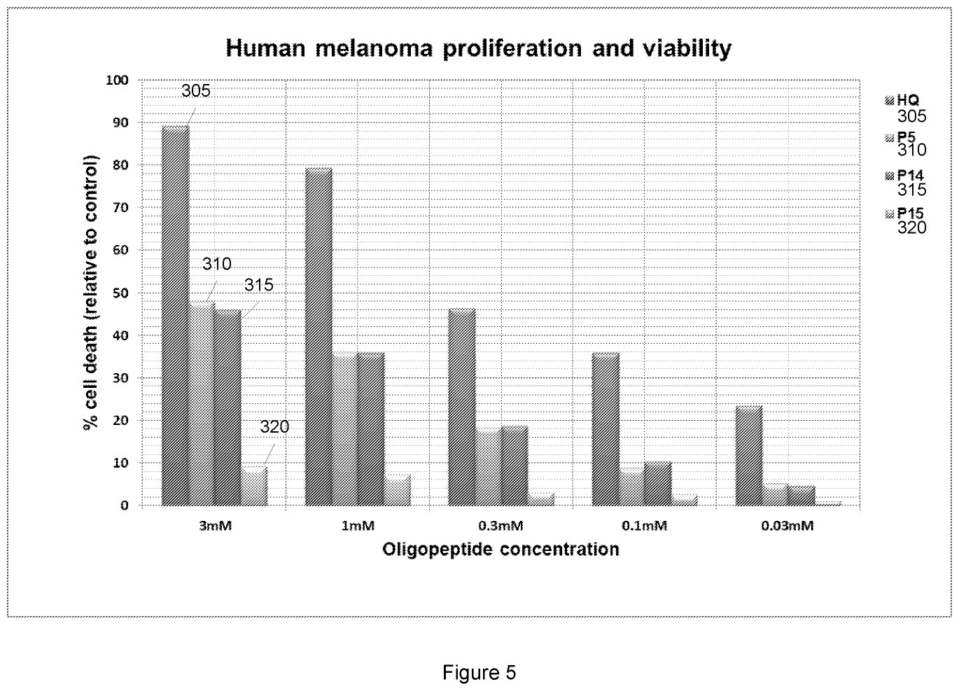

FIG. 4 also shows that the cytotoxic effect was dose dependent. FIG. 5 shows the cytotoxic effects on human melanoma cells. At a concentration of 1 millimolar, P5 and P14 showed nearly similar cytotoxicity of 36 percent compared to nearly 80 percent cytotoxicity of HQ.

Tables 1,2, and 3 show the effects of P5 and P14 on the vitality and viability of human melanocytes and fibroblasts compared to HQ. After 72 hours of incubation with 1 millimolar P5 and P14, cell death was 9 percent and 9 percent, for melanocytes, respectively, and 3 percent and 6 percent for fibroblasts, respectively. On the other hand, 1 millimolar HQ resulted in 100 percent cell death in both melanocytes and fibroblasts after 72-hour incubation. The tables clearly show the indiscriminate cytotoxicity of HQ compared to the selective effects of oligopeptides P5 and P14.

The results observed in the MTT assay appeared consistent with our preliminary in silico modeling data. For example, P15 showed weak in silico binding when docked in the ATP pocket of either B-RAFV600E or C-RAF, and weak effects on the proliferation and viability of melanoma cells (FIGS. 4 and 5). On the other hand, oligopeptides P5 and P14 showed free binding energies that were comparable with those of sorafenib and PLX4032. Taken together, our findings support the use of molecular modeling as a screening tool to identify oligopeptides with potential cytotoxic effects towards human metastatic melanoma lines in vitro.

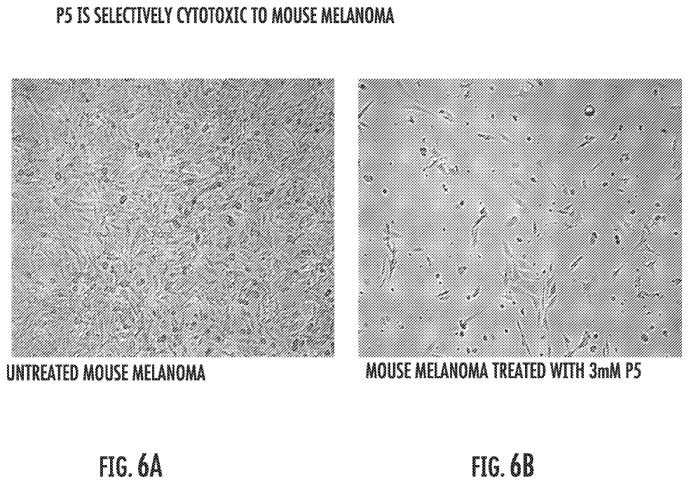

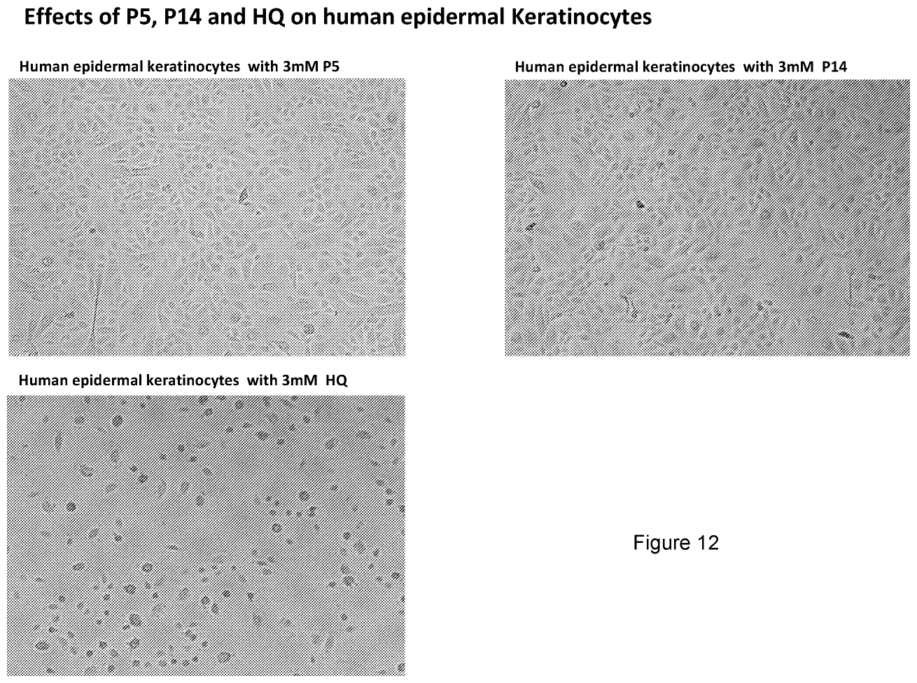

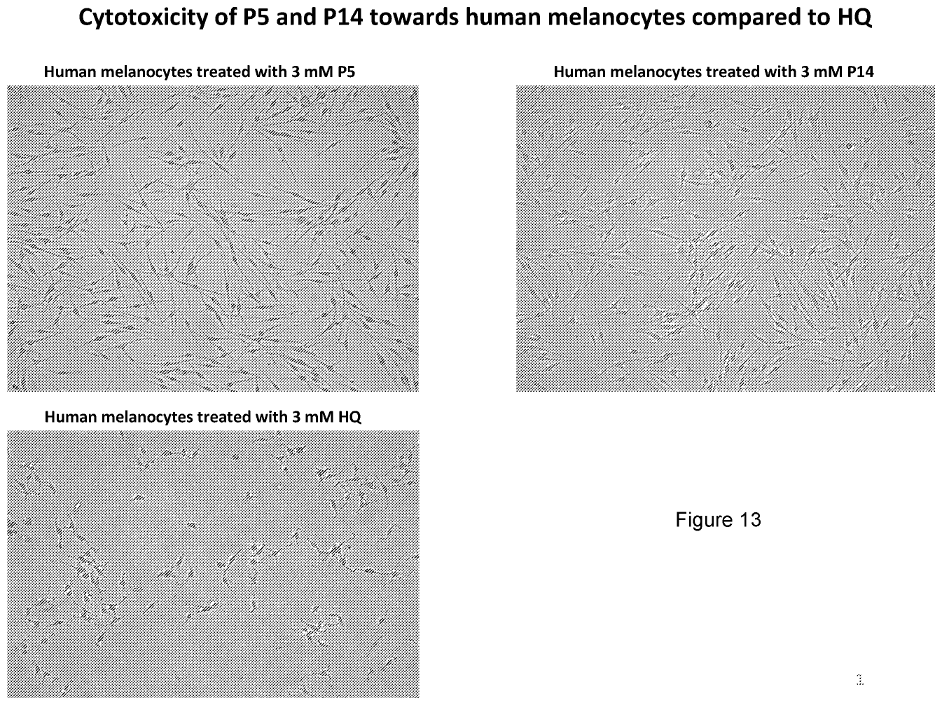

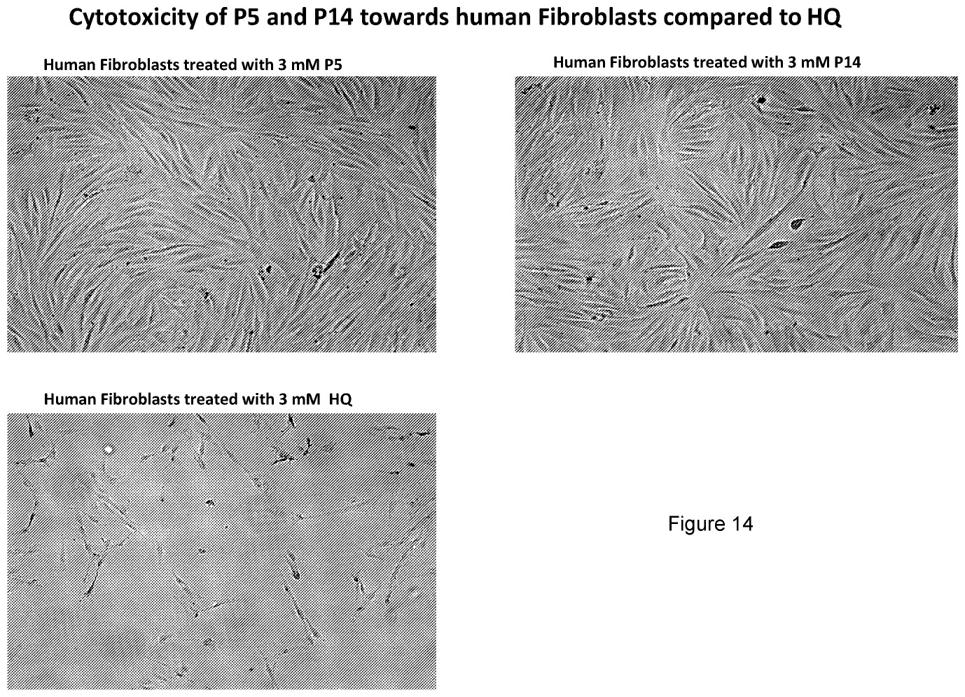

FIGS. 6-11 confirm the cytotoxic effects that P5, P14, and HQ exert on mouse and human melanoma cells. However, FIGS. 12-14 demonstrate the selective cytotoxicity of P5 and P14 compared to the indiscriminate cytotoxicity of HQ towards healthy normal skin cells like epidermal keratinocytes (FIG. 12), melanocytes (FIG. 13), and fibroblasts (FIG. 14).

### Discussion

Oligopeptides P5 and P14 are capable of selectively killing melanoma cells while exhibiting very little to no cytotoxic effects towards normal human melanocytes, keratinocytes, and fibroblasts which are the major constituents of the skin. HQ on the other hand is a potent nonselective cytotoxic agent against many cell lines. We used HQ as a positive control because of its known cytotoxic effects against some cancer cell lines. Oligopeptide P15 was used as a negative control to highlight the selectivity of P5 and P14 against melanoma cells and to validate the output of the in silico molecular modeling. P15 showed weak in silico binding when docked in the ATP pocket of either B-RAFV600E or C-RAF, and weak cytotoxicity for P15 (FIGS. 4 and 5). On the other hand, oligopeptides P5 and P14 showed free binding energies that were comparable with those of sorafenib and PLX4032.

Our findings support the use of molecular modeling as a screening tool to identify oligopeptides with potential cytotoxic effects towards human metastatic melanoma lines in vitro. Unlike HQ, P5 and P14 exhibited selective cytotoxicity towards melanoma cells with minimal or no cytotoxic effects towards normal skin cells, melanocytes, keratinocytes, and fibroblasts. MAPK inhibition has been shown to result in dephosphorylation of the pro-apoptotic Bcl-2 family members, Bad, and Bim. This, in turn, leads to activation of the caspase pathway, and ultimately, the demise of melanoma cells through the induction of apoptosis.

Although expectations were high when sorafenib, the first RAF kinase-targeting drug, reached clinical trials, initial studies in melanoma patients were disappointing and did not show the anticipated single agent efficacy. The problem with sorafenib and similar development candidates is their lack of specificity. Furthermore, melanoma tumors harboring the BRAFV600E mutation were found to be resistant to sorafenib treatment, implying only tumors with low B-RAF activity were sensitive to the drug. This phenomenon has been attributed to dependence of these tumors on C-RAF for MEK activity and B-cell lymphoma 2-mediated cell survival. Thus, since greater than 90 percent of melanomas exhibit B-RAF hyperactivity, only a small percentage of tumors will respond to sorafenib. Interestingly, sorafenib was later shown to more potently inhibit C-RAF kinase.

More recently, the resolution of the B-RAF crystal structure has led to the development of more specific B-RAF inhibitors such as PLX4032. This drug induced a dramatic response rate in phase I trials, validating B-RAF as a clinically relevant target. Unfortunately, it has recently been shown that elevated CRAF gene expression mediates resistance to B-RAF inhibitors in BRAFV600E-mutant melanoma cells. Furthermore, in RAS-mutant melanoma cells, C-RAF is essential because oncogenic RAS signals through this kinase rather than B-RAF. These findings suggest that highly targeted and specific B-RAF inhibitors may be ineffective against both RAS-mutant melanomas and BRAF-mutant melanomas with high C-RAF activity. In other words, the above findings point to the need for pan-RAF inhibition.

Of equal significance is the discovery that inhibition of BRAFV600E increased levels of melanocyte differentiation antigens (MDAs), resulting in improved recognition by antigen- specific T lymphocytes. This is supported by the finding that loss of antigen expression led to relapse after immunotherapy. Melanocytic cells express a set of genes specific to that lineage. However, with respect to immunotherapy, several melanocytic gene products, including MART1 and gp100, have been directly shown to function as target antigens for cytotoxic T lymphocytes. This has led some investigators to hypothesize that reduced MDA expression by melanoma cells facilitates evasion of tumor surveillance in vivo. Thus, blockade of MAPK signaling via a specific BRAFV600E inhibitor with subsequent elevation of MDA expression may hold great potential as an immunotherapeutic approach for advanced melanoma.

Based on our data and previous studies, we hypothesize that oligopeptide treatment enhances melanoma killing by inhibiting B-RAFV600E- and C-RAF-dependent MAPK signaling, leading to unopposed caspase activity and subsequent apoptosis, as well as by increasing MDA expression, resulting in the potential for improved recognition by MART1-specific and gp100-specific autologous T lymphocytes. Moreover, and to help address the urgent need for an effective preventative treatment for melanoma, our approach would be to target high risk groups with a non-invasive and non-surgical prophylactic topical treatment formulated with P5 or P14. These peptides may prove potential, cost effective, and efficacious treatment for early stage melanoma in situ (stage 0 melanoma), carcinoma in situ (basal and squamous cell carcinoma) and the elimination of any new dysplastic nevi on sun exposed areas of the skin.

TABLE 1A
Peptide Toxicity on Human Primary Melanocytes Using Trypan Blue
Day 1, P5, PLG

| Day 1 | [μM] | % toxicity | SEM |
|---|---|---|---|
| | 30 | 0.1 | 0 |
| P5 | 100 | 0.1 | 0 |
| PLG | 300 | 2.2 | 0.91 |
| | 1000 | 7.8 | 0.91 |
| | 3000 | 13.3 | 1.57 |

TABLE 1B
Peptide Toxicity on Human Primary Melanocytes Using Trypan Blue
Day 3, P5, PLG

| Day 3 | [μM] | % toxicity | SEM |
|---|---|---|---|
| | 30 | 0.1 | 0 |
| P5 | 100 | 0.1 | 0 |
| PLG | 300 | 2.4 | 0.33 |
| | 1000 | 8.7 | 0.56 |
| | 3000 | 13.5 | 1.06 |

TABLE 1C
Peptide Toxicity on Human Primary Melanocytes Using Trypan Blue
Day 6, P5, PLG

| Day 6 | [μM] | % toxicity | SEM |
|---|---|---|---|
| | 30 | 0.1 | 0 |
| P5 | 100 | 0.1 | 0 |
| PLG | 300 | 2.7 | 1.09 |
| | 1000 | 9.3 | 1.03 |
| | 3000 | 14.5 | 1.26 |

TABLE 1D
Peptide Toxicity on Human Primary Melanocytes Using Trypan Blue
Day 1, P14, RRFVLL

| Day 1 | [μM] | % toxicity | SEM |
|---|---|---|---|
| | 30 | 0.1 | 0 |
| P14 | 100 | 0.1 | 0 |
| RRFVLL | 300 | 0.1 | 0 |
| | 1000 | 6.3 | 0.26 |
| | 3000 | 7.9 | 0.32 |

TABLE 1E
Peptide Toxicity on Human Primary Melanocytes Using Trypan Blue
Day 3, P14, RRFVLL

| Day 1 | [μM] | % toxicity | SEM |
|---|---|---|---|
| | 30 | 0.1 | 0 |
| P14 | 100 | 0.1 | 0 |
| RRFVLL | 300 | 0.1 | 0 |
| | 1000 | 5 | 0.37 |
| | 3000 | 8.4 | 0.57 |

TABLE 1F
Peptide Toxicity on Human Primary Melanocytes Using Trypan Blue
Day 6, P14, RRFVLL

| Day 1 | [μM] | % toxicity | SEM |
|---|---|---|---|
| | 30 | 0.1 | 0 |
| P14 | 100 | 0.1 | 0 |
| RRFVLL | 300 | 0.1 | 0 |
| | 1000 | 7.9 | 0.41 |
| | 3000 | 11.6 | 0.87 |

TABLE 2A
Peptides P5 and P14 toxicity towards
Human Fibroblasts Using Trypan Blue
Day 1, P5, PLG

| Day 1 | [μM] | % toxicity | SEM |
|---|---|---|---|
| | 10 | 0.1 | 0 |
| P5 | 30 | 0.1 | 0 |
| PLG | 100 | 0.1 | 0 |
| | 300 | 0.1 | 0 |
| | 1000 | 2.1 | 0.32 |

TABLE 2B
Peptides P5 and P14 toxicity towards
Human Fibroblasts Using Trypan Blue
Day 3, P5, PLG

| Day 3 | [μM] | % toxicity | SEM |
|---|---|---|---|
| | 10 | 0.1 | 0 |
| P5 | 30 | 0.1 | 0 |
| PLG | 100 | 0.1 | 0 |
| | 300 | 0.5 | 0 |
| | 1000 | 2.6 | 1.02 |

TABLE 2C
Peptides P5 and P14 toxicity towards
Human Fibroblasts Using Trypan Blue
Day 6, P5, PLG

| Day 6 | [μM] | % toxicity | SEM |
|---|---|---|---|
| | 10 | 0.1 | 0 |
| P5 | 30 | 0.1 | 0 |
| PLG | 100 | 0.1 | 0 |
| | 300 | 1.1 | 0 |
| | 1000 | 3.9 | 0.32 |

TABLE 2D
Peptides P5 and P14 toxicity towards
Human Fibroblasts Using Trypan Blue
Day 1, P14, RRFVLL

| Day 1 | [μM] | % toxicity | SEM |
|---|---|---|---|
| | 10 | 0.1 | 0 |
| P14 | 30 | 0.1 | 0 |
| RPFVLL | 100 | 0.1 | 0 |
| | 300 | 4.3 | 0.43 |
| | 1000 | 6.3 | 0.48 |

TABLE 2E
Peptides P5 and P14 toxicity towards
Human Fibroblasts Using Trypan Blue
Day 3, P14, RRFVLL

| Day 3 | [μM] | % toxicity | SEM |
|---|---|---|---|
| | 10 | 0.1 | 0 |
| P14 | 30 | 0.1 | 0 |
| RPFVLL | 100 | 0.1 | 0 |
| | 300 | 3.8 | 0.27 |
| | 1000 | 5.9 | 0.17 |

TABLE 2F
Peptides P5 and P14 toxicity towards
Human Fibroblasts Using Trypan Blue
Day 6, 14, RRFVLL

| Day 6 | [μM] | % toxicity | SEM |
|---|---|---|---|
| | 10 | 0.1 | 0 |
| P14 | 30 | 0.1 | 0 |
| RPFVLL | 100 | 0.1 | 0 |
| | 300 | 4.0 | 0.13 |
| | 1000 | 6.9 | 0.41 |

TABLE 3A
Hydroquinone is toxic to Human Primary Melanocytes Using Trypan Blue
Day 1, HQ

| Day 1 | [μM] | % toxicity | SEM |
|---|---|---|---|
| | 10 | 27 | 3.3 |
| HQ | 30 | 41 | 2.8 |
| | 100 | 98 | 1.1 |
| | 300 | 100 | 0 |
| | 1000 | 100 | 0 |

TABLE 3B
Hydroquinone is toxic to Human Primary Melanocytes Using Trypan Blue
Day 3, HQ

| Day 3 | [μM] | % toxicity | SEM |
|---|---|---|---|
| | 10 | 29 | 4.6 |
| HQ | 30 | 53 | 5.2 |
| | 100 | 100 | 0 |
| | 300 | 100 | 0 |
| | 1000 | 100 | 0 |

TABLE 3C
Hydroquinone is toxic to Human Primary Melanocytes Using Trypan Blue
Day 6, HQ

| Day 6 | [μM] | % toxicity | SEM |
|---|---|---|---|
| | 10 | 35 | 3.8 |
| HQ | 30 | 60 | 2.6 |
| | 100 | 100 | 0 |
| | 300 | 100 | 0 |
| | 1000 | 100 | 0 |

TABLE 3D
Hydroquinone is also toxic towards Human Fibroblasts
Day 1, HQ

| Day 1 | [μM] | % toxicity | SEM |
|---|---|---|---|
| | 30 | 3 | 0 |
| HQ | 100 | 17 | 1 |
| | 300 | 27 | 1.1 |
| | 1000 | 100 | 0 |
| | 3000 | 100 | 0 |

TABLE 3E
Hydroquinone is also toxic towards Human Fibroblasts
Day 3 HQ

| Day 3 | [μM] | % toxicity | SEM |
|---|---|---|---|
| | 30 | 9 | 0 |
| HQ | 100 | 20 | 1.2 |
| | 300 | 44 | 2.4 |
| | 1000 | 100 | 0 |
| | 3000 | 100 | 0 |

TABLE 3F
Hydroquinone is also toxic towards Human Fibroblasts
Day 1, HQ

| Day 6 | [μM] | % toxicity | SEM |
|---|---|---|---|
| | 30 | 15 | 1.2 |
| HQ | 100 | 30 | 1.1 |
| | 300 | 54 | 3.1 |
| | 1000 | 100 | 0 |
| | 3000 | 100 | 0 |

The tables clearly show the indiscriminate cytotoxicity of HQ compared to the selective effects of oligopeptides P5 and P14.

FIGS. 4-9 demonstrably confirm the cytotoxic effects that P5, P14, and HQ exert on mouse and human melanoma cells.

FIG. 4 shows results where P5 is selectively cytotoxic to mouse melanoma. In A, untreated mouse melanoma is shown. In B, mouse melanoma treated with 3 millimolar P5 is shown.

FIG. 5 shows results where P5 is selectively cytotoxic to human melanoma. In A, untreated human melanoma is shown. In B, human melanoma treated with 3 millimolar P5 is shown.

FIG. 6 shows results where P14 is selectively cytotoxic to human melanoma. In A, untreated human melanoma is shown. In B, human melanoma treated with 3 millimolar P14 is shown.

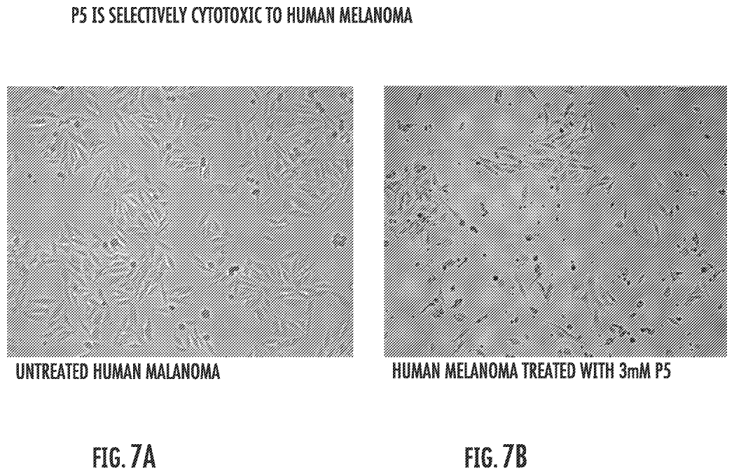

FIG. 7 shows results where P14 is selectively cytotoxic to mouse melanoma. In A, untreated mouse melanoma is shown. In B, mouse melanoma treated with 3 millimolar P14 is shown.

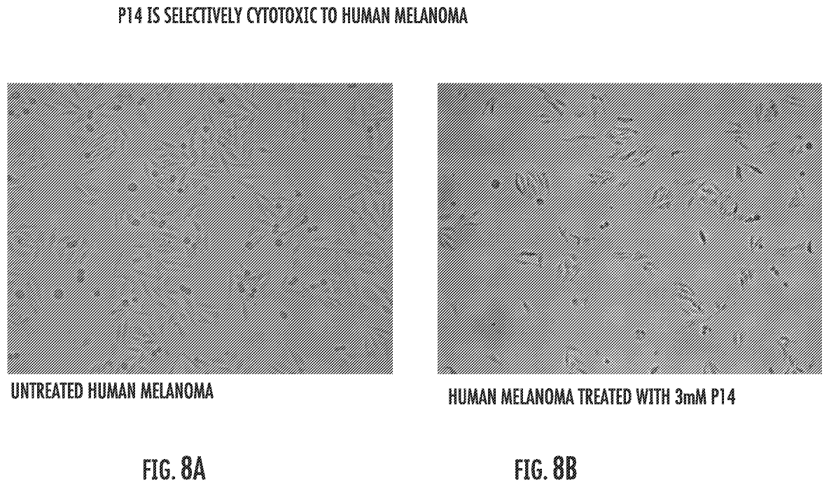

FIG. 8 shows results where HQ is a potent nonselective cytotoxic agent against human melanoma cells. In A, untreated human melanoma is shown. In B, Human melanoma treated with 3 millimolar HQ is shown.

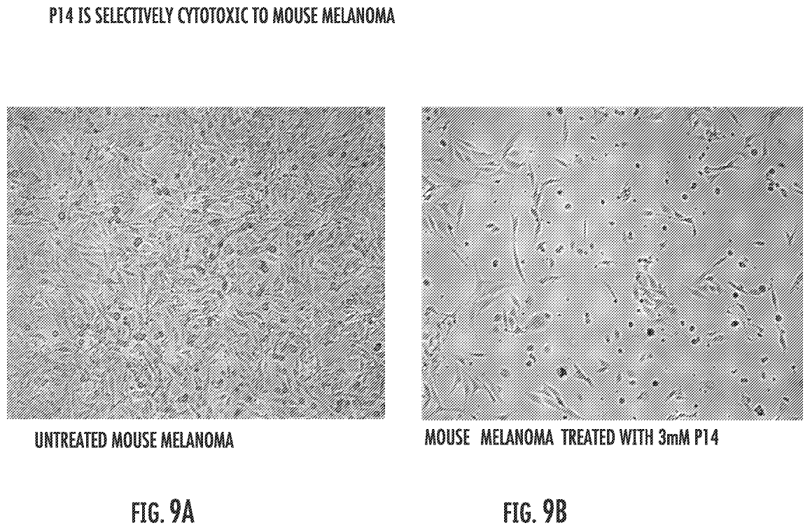

FIG. 9 shows results where cytotoxicity of HQ towards mouse melanoma. In A, untreated B16 mouse melanoma is shown. In B, B16 mouse melanoma treated with 1 millimolar HQ is shown.

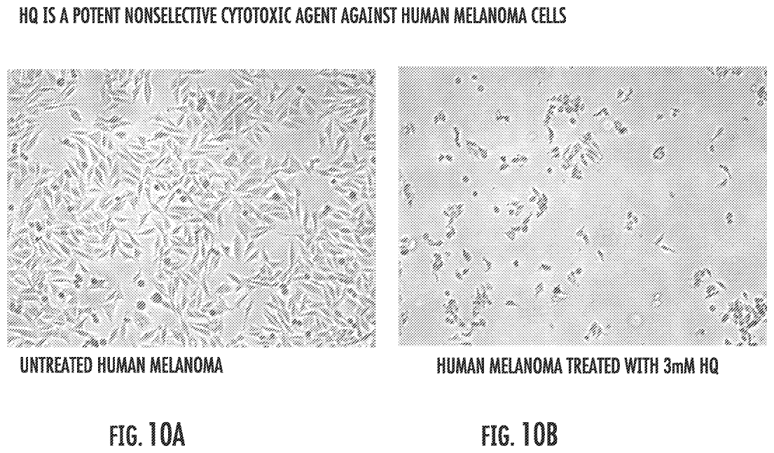

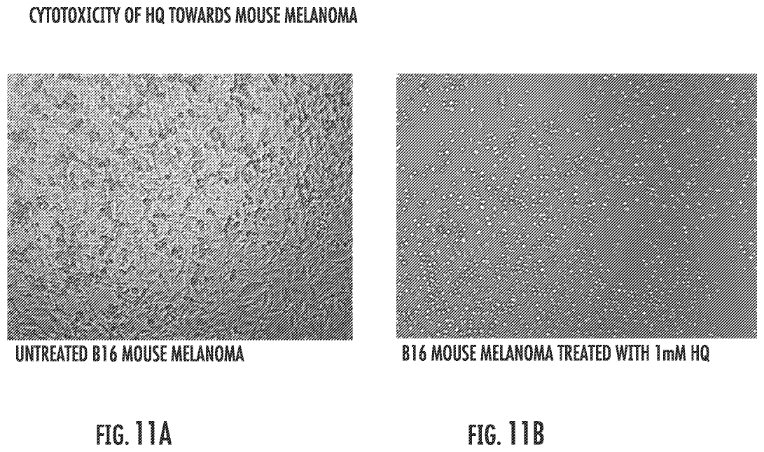

FIGS. 10, 11, and 12 are of extreme importance because they point out the selective cytotoxicity of P5 and P14 compared to the indiscriminate toxicity of HQ towards healthy normal skin cells like epidermal keratinocytes in FIG. 10, melanocytes in FIG. 11 and fibroblasts in FIG. 12.

FIG. 10 shows effects of P5, P14, and HQ on human epidermal keratinocytes. The figures show human epidermal keratinocytes with 3 millimolar P5, human epidermal keratinocytes with 3 millimolar P14, and human epidermal keratinocytes with 3 millimolar HQ.

FIG. 11 shows cytotoxicity of P5 and P14 towards human melanocytes compared to hydroquinone. The figures show human melanocytes treated with millimolar P5, human melanocytes treated with 3 millimolar P14, and human melanocytes treated with millimolar HQ.

FIG. 12 shows cytotoxicity of P5 and P14 towards human fibroblasts compared to hydroquinone. The figures show human fibroblasts treated with 3 millimolar P5, human fibroblasts treated with 3 millimolar P14, and human fibroblasts treated with 3 millimolar HQ.

Based on the above facts, and to help address the urgent need for an effective preventative treatment for melanoma, our approach would be to target high risk groups with a non-invasive and non-surgical prophylactic treatment with our oligopeptides, P5 and P14 that have proven to be selectively cytotoxic towards melanoma cell lines. Screening of high-risk patients is cost-effective and likely to be associated with an improved survival. Individuals with xeroderma pigmentosum, giant congenital nevi, immunosuppression, familial atypical multiple mole and melanoma syndrome, unusual-appearing nevi, numerous (greater than 50) nevi, changing nevi, and a family history of melanoma and men older than 50 years should receive complete baseline and periodic follow-up skin examinations by a physician.

Moreover, these peptides may prove potential, cost effective and efficacious treatment for early stage melanoma in situ (stage 0 melanoma,), carcinoma in situ (Basal and Squamous cell carcinoma) and the elimination of any new dysplastic nevi on sun exposed areas of the skin. Embodiments can be used to induce cytotoxicity of pathologic melanocytes including mutated melanocytes, premalignant melanocytes, and melanoma cells

This description of the invention has been presented for the purposes of illustration and description. It is not intended to be exhaustive or to limit the invention to the precise form described, and many modifications and variations are possible in light of the teaching above. The embodiments were chosen and described in order to best explain the principles of the invention and its practical applications. This description will enable others skilled in the art to best utilize and practice the invention in various embodiments and with various modifications as are suited to a particular use. The scope of the invention is defined by the following claims.

The invention claimed is:

1. A method of treating a subject by inducing cytotoxicity of pathologic melanocytes including mutated melanocytes, premalignant melanocytes, and melanoma cells, the method comprising administering to a subject in need thereof a composition comprising an effective amount of one or more oligopeptides, wherein the one or more oligopeptides consist of,

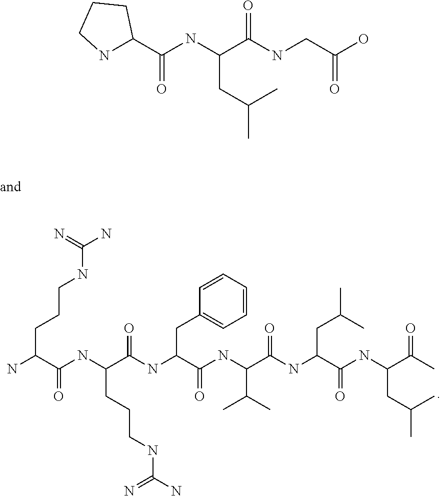 [SMILES: CC(C)CC(NC(=O)C1CCCN1)C(=O)NCC(=O)O ; CC(=O)C(CC(C)C)NC(=O)C(CC(C)C)NC(=O)C(NC(=O)C(Cc1ccccc1)NC(=O)C(CCCNC(=N)N)NC(=O)C(N)CCCNC(=N)N)C(C)C]

2. The method of claim 1 wherein the pathologic melanocytes are mammalian cells.

3. The method of claim 2 wherein the pathologic melanocytes are human cells.

4. An oligopeptide consisting of:

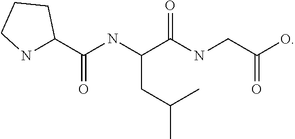

5. An oligopeptide consisting of:

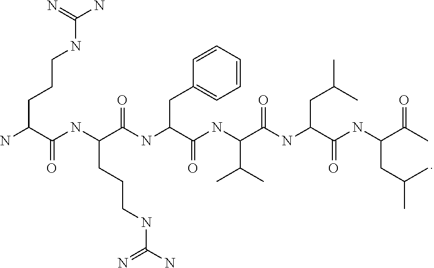 [SMILES: CC(=O)C(CC(C)C)NC(=O)C(CC(C)C)NC(=O)C(NC(=O)C(Cc1ccccc1)NC(=O)C(CCCNC(=N)N)NC(=O)C(N)CCCNC(=N)N)C(C)C]

6. A therapeutic agent for treating pathogenic melanocytes comprising one or more oligopeptides consisting of: 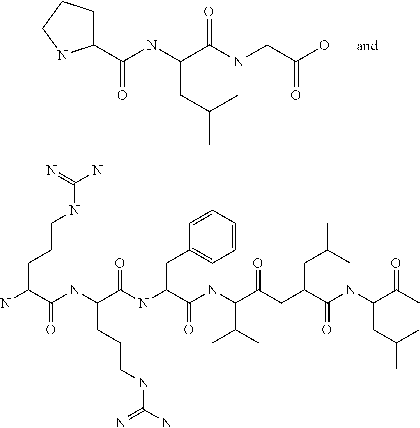 [SMILES: CC(C)CC(NC(=O)C1CCCN1)C(=O)NCC(=O)O ; CC(=O)C(CC(C)C)NC(=O)C(CC(=O)C(NC(=O)C(Cc1ccccc1)NC(=O)C(CCCNC(=N)N)NC(=O)C(N)CCCNC(=N)N)C(C)C)CC(C)C]
